(19) United States
(12) Patent Application Publication
(10) Pub. No.: US 2006/0110466 A1
(43) Pub. Date: May 25, 2006

(54) Natural water wipes

(76) Inventor: Anna L. Haizlip, Kernersville, NC (US)

Correspondence Address: Anna L. Haizlip, 3679 SHEPPARD HILL ROAD, Kernersville, NC, 27284, US

(21) Appl. No.: 10/994,953

(22) Filed: Nov. 22, 2004

(51) Int. Cl. A61K 33/00

(52) U.S. Cl. 424/600

(57) ABSTRACT

The natural water wipe is unlike others in this category because it is devoid of any added substance, active ingredient or solution. Water will be the only natural ingredient used to avoid irritation or allergic reactions to the skin or to any body parts such as the mouth, eyes, ears or nose, or arm pits. The wipe is a fragrance and odor free product. Other products are heavily laden with solutions that produce intentionally induced fragrances or smells or odors that result from the overall composition of the product itself.

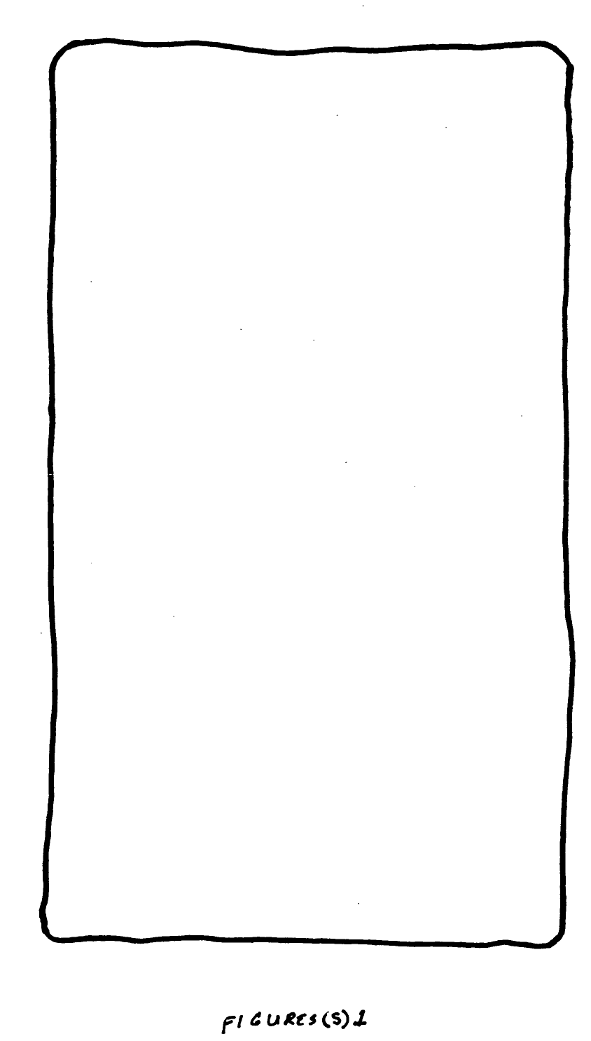

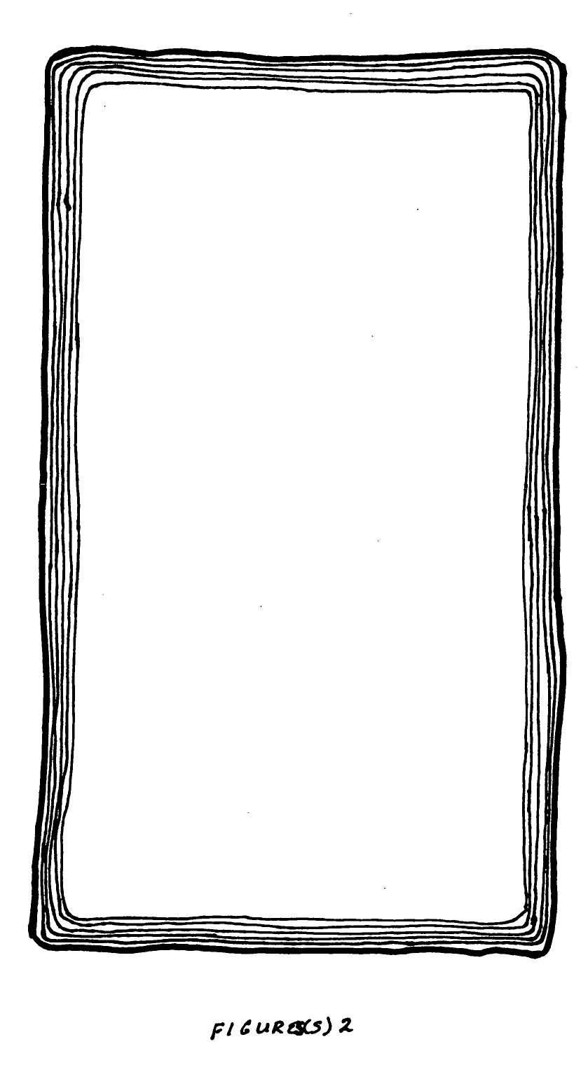

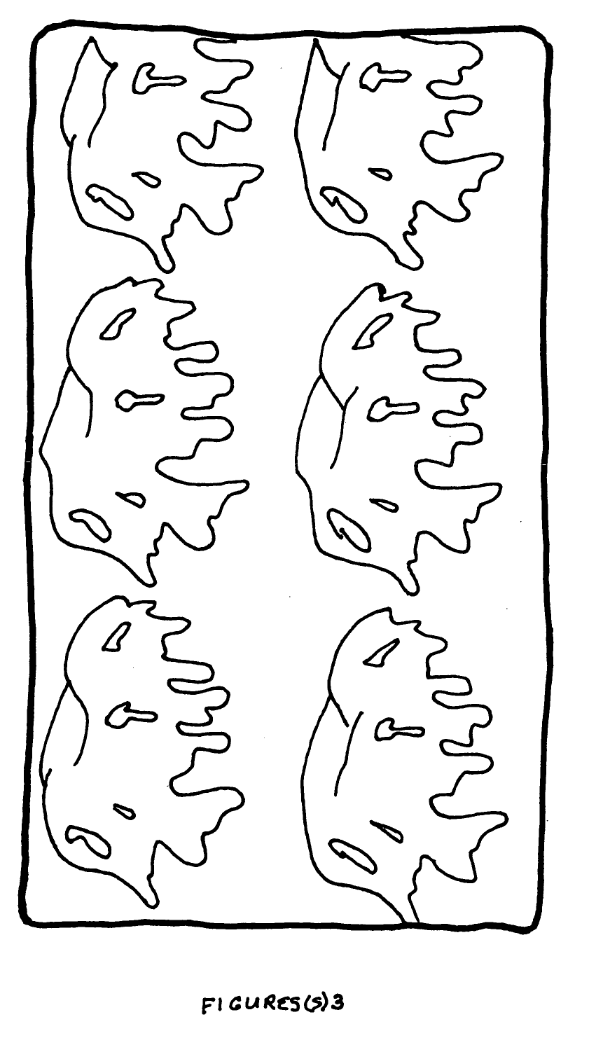

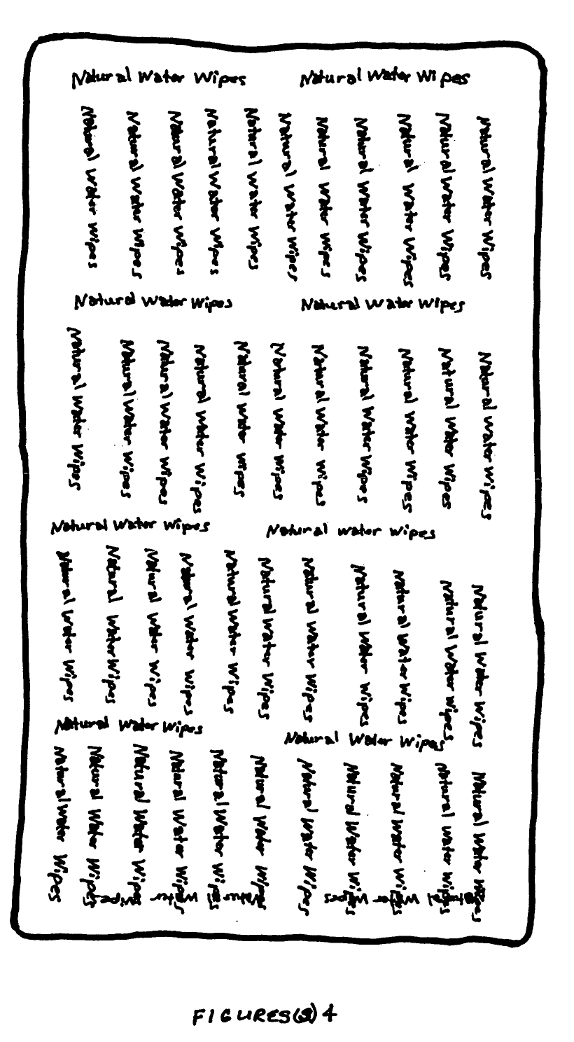

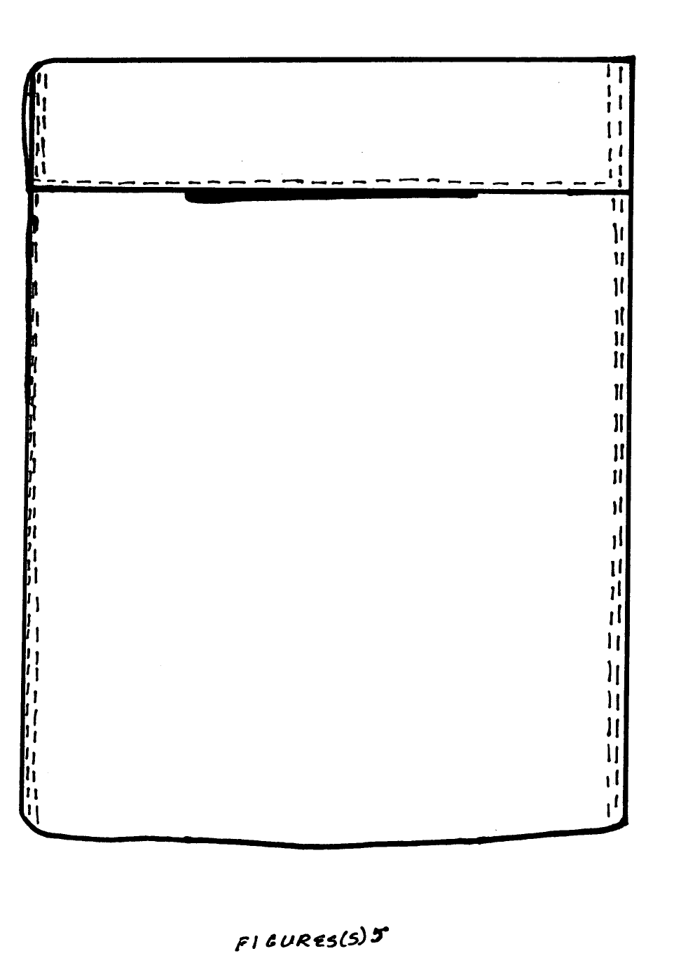

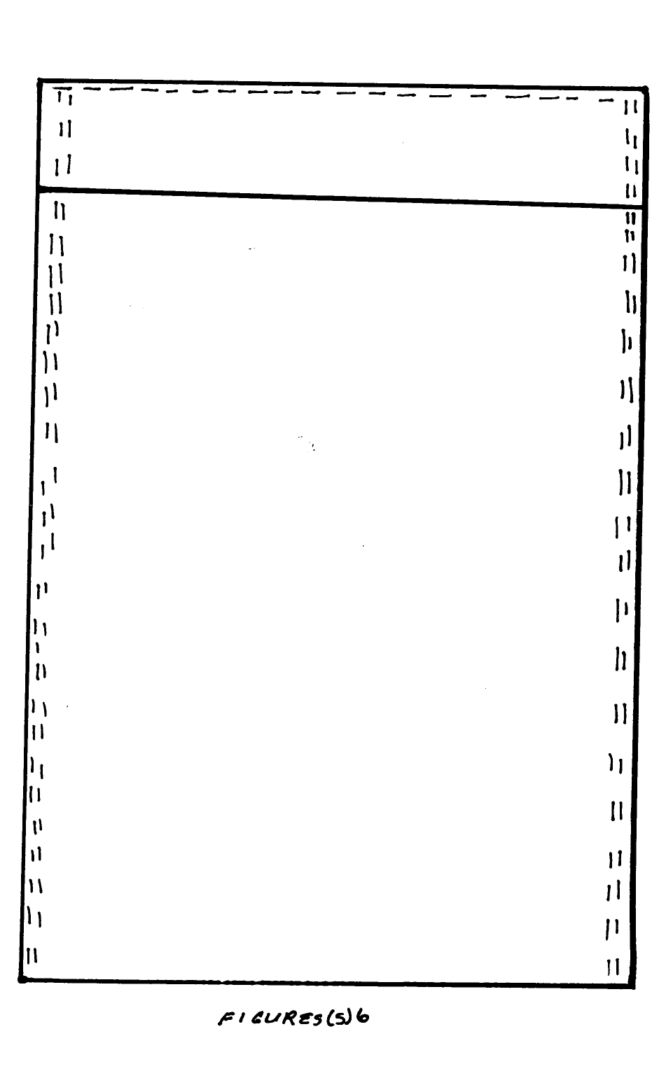

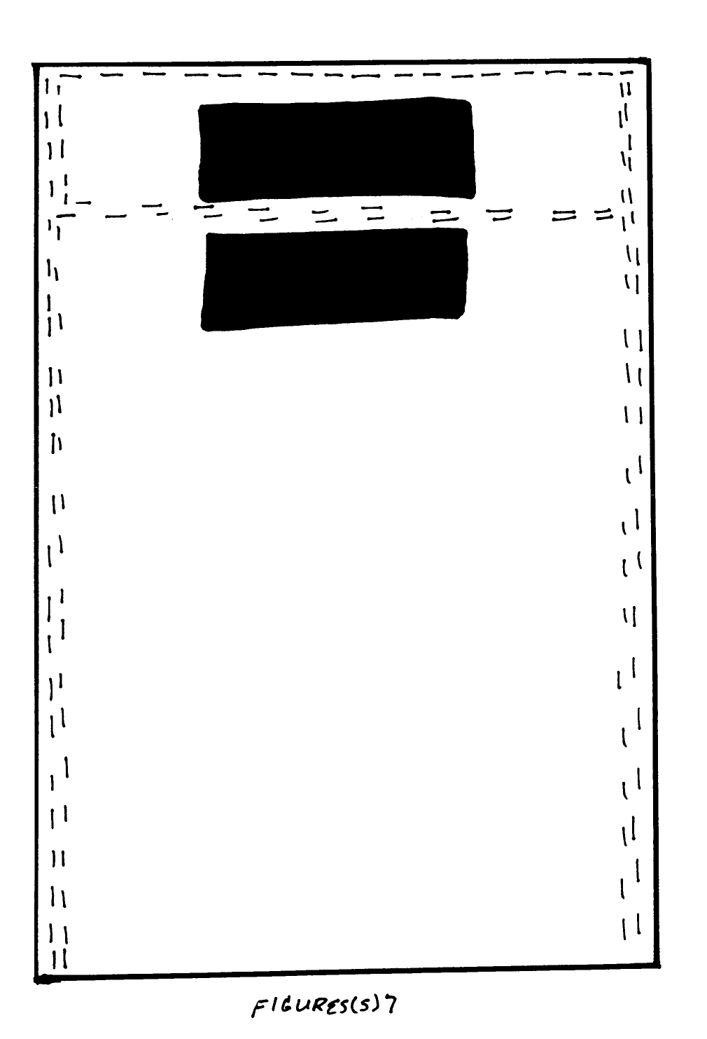

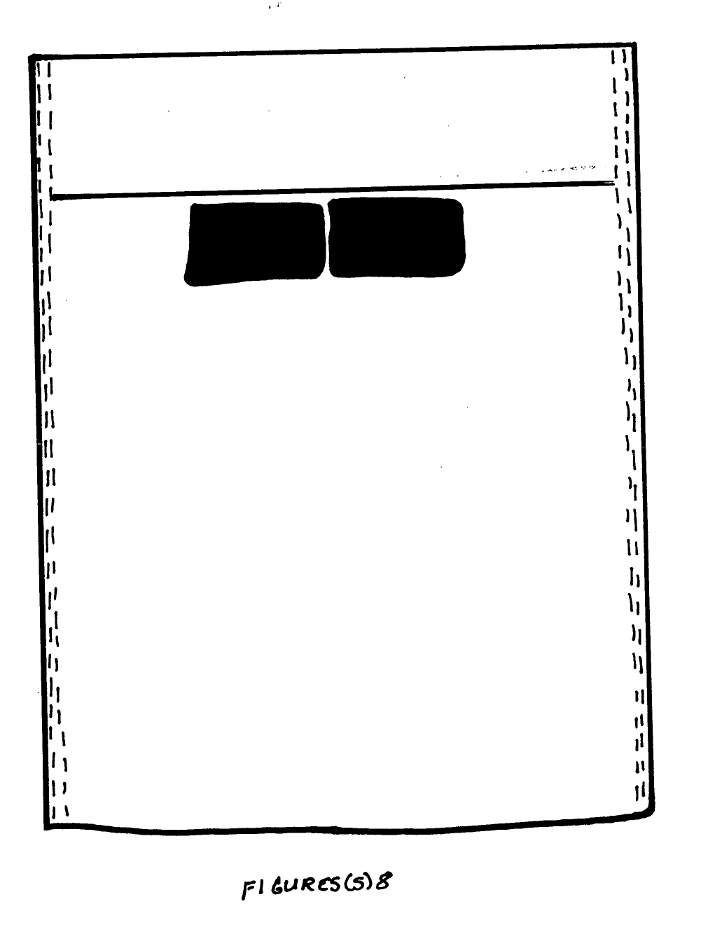

### BACKGROUND TO THE INVENTION

The invention is related to the field of “wipes” but using water as a natural agent to induce a cooling effect after exertion, exercise, or any temperature related condition. It is primarily designed to serve as a cooling agent to be applied to the various parts of the body.

Additionally, it may also serve as a cooling or cleaning instrument for wounds or cuts. The unique composition of the natural water wipe assures safety and appropriateness for use in situations requiring initial cleansing of cuts or wounds. The natural water wipes will come with a pouch that can be attached to clothing or pants by a velcro loop.

### PRIOR ART

Wipes are used for a variety of purposes such as cleansing, moisturizing, and lubricating as well as a number of other activities. The nature of most wipes use added agents, chemicals, substances or solutions to bring about the desired result of the wipe itself. Unlike these agents, this invention employs natural water as the only added component to the product.

### BRIEF SUMMARY TO THE INVENTION

The natural water wipe is a 6″×12″×½″ cloth infused with water droplets. The invention provides a natural water wipe devoid of any added substance, active ingredient or solution. Unlike other products the water as a natural ingredient is used to avoid irritation or allergic reactions to the skin or to any body parts such as the mouth, eyes, ears or nose, or arm pits. The composition of the wipe also affords users a fragrance and odor free product. Other products are heavily laden with solutions that produce intentionally induced fragrances or smells or odors that result from the overall composition of the product itself. The wipe will be disposable and flushable. An accompanying carrying pouch is provided for transporting and preserving natural water wipe moisture.

### BRIEF DESCRIPTION OF THE SEVERAL VIEWS OF THE DRAWING

1. Overview perspective of the entire natural water wipe

2. A cross-sectional view of the layers of the water wipe

3. A diagram perspective of the textured or fabric of the water wipe

4. A diagram of the flow of water droplets in water wipe

5. Sketch of the name imprint on the top and bottom layer of the wipe

### DETAILED DESCRIPTION OF THE INVENTION

Natural water wipes are 6″×12″×½″ cloths infused with/ natural water. The wipes serve as a cooling or cleansing piece to be used during such activities, as exercise, exertion, or any heat related condition or as a cleansing item for cuts or wounds. Cloth like material will release moisture for cooling the body after daily exertion thus renewing the body temperature to its normal level. Five layers of the wipe will be laid in stacks and pressed to support the moisture of the wipe. Drawing number one show the shape, width and length of the wipe. Drawing number two shows the number of layers which can five or more. Drawing number three illustrates the design and texture of the moisture within the cloth like material. Drawing number four shows the outside covering of the wipe with the name natural water wipe name imprint. Drawing number five shows a front view of the natural water wipe pouch with the top down. Drawing number six shows the front view of the pouch with the top closed by attached velcro. Drawing number seven illustrates the back side view of natural water wipe pouch. Drawing number eight illustrates the back side view with the attached velcro loops.

I claim my invention:

1. Will use only natural water as an ingredient unlike other products that use other solutions or agents.
Will not irritate or cause allergic reactions to skin or body parts. Serve as a cooling or cleansing product.
Is fragrance and odor free.
